(19) United States
(12) Patent Application Publication
(10) Pub. No.: US 2022/0273673 A1
(43) Pub. Date: Sep. 1, 2022

(54) BONE-BINDING COMPOUNDS

(71) Applicant: BRIGHAM YOUNG UNIVERSITY, Provo, UT (US)

(72) Inventors: Paul B. SAVAGE, Mapleton, UT (US); Aaron SCHINDELER, New South Wales NSW (AU); Peter VALTCHEV, New South Wales NSW (AU); Sumedh KAMBLE, New South Wales NSW (AU)

(73) Assignee: BRIGHAM YOUNG UNIVERSITY, Provo, UT (US)

(21) Appl. No.: 17/629,058

(22) PCT Filed: Jul. 24, 2020
(86) PCT No.: PCT/US2020/043563

§ 371 Date: Jan. 21, 2022

Related U.S. Application Data
(60) Provisional application No. 62/878,199, filed on Jul. 24, 2019.

(51) Int. Cl. A61K 31/56; A61K 31/663; A61K 47/54; A61K 47/55; A61P 19/08; A61P 35/00

(52) U.S. Cl. CPC A61K 31/56; A61K 31/663; A61K 47/548; A61K 47/552; A61P 19/08; A61P 35/00; A61B 2017/00889

(57) ABSTRACT

Disclosed herein are compounds having the following Formula (I) or a pharmaceutically acceptable salt form thereof: B-L-C (I) wherein B is a bone-binding moiety; L is a linker; and C is a cationic steroid antimicrobial (CSA) moiety, pharmaceutical compositions comprising the compounds, and methods of using the compounds or pharmaceutical compositions for the treatment of an infection or osteomyelitis in a bone of a subject, promotion of bone formation in a subject, or treatment of bone cancer or metastatic bone cancer in a subject.

### BACKGROUND

The present disclosure relates to antibiotic compounds and, in particular, antibiotic compounds which bind to bone and are useful in the treatment of bone infections.

Any discussion of the prior art throughout the specification should in no way be considered as an admission that such prior art is widely known or forms part of the common general knowledge in the field.

Infection of open fractures is a major cause of morbidity and mortality worldwide (Tay et al. Injury. 2014. 45: 1653-1658; Wu. Othrop Res Rev. 2013. 5: 21-33). Even when wounds are not visibly contaminated, open fractures generally have an increased risk of becoming infected compared with equivalent closed fractures. Bone infections can also arise in the absence of a fracture. In osteomyelitis, for example, bacteria can reach the bone via the bloodstream or by spreading from nearby tissue, such as from diabetic foot infections. Infections can also originate in the bone following an injury which exposes the bone to bacteria. Additionally, implants can lead to bone infections.

Treating bone infections is complicated by their relatively low vascularization and their location beneath soft tissue in the body. High doses of systemic drugs and long treatment regimens may be required to provide effective concentrations of the drug at the site of infection. However, this can cause harmful side effects and thereby limit the use of certain antibiotics. While local delivery of antibiotics to surgical sites has been explored, this approach is still in the experimental phase and requires an open surgical site. Moreover, widespread antibiotic use over long periods of time has resulted in the development of antibiotic resistant strains of bacteria. The incidence of community-acquired antibiotic-resistant strains such as methicillin-resistant Staphylococcus aureus, methicillin-resistant Staphylococcus epidermidis, vancomycin-resistant Staphylococcus spp., and tobramycin-resistant Pseudomonas aeruginosa is growing, with rates of up to 25% reported in the United States (Chen et al. Clin Orthop Relat Res. 2013. 471: 3135-3140; Stevens. Curr Opin Infect Dis. 2003. 16: 189-191).

In this context, there is a need for new antibiotics and antibiotic therapies, particularly for orthopedic applications.

### SUMMARY

The present disclosure relates to antibiotic compounds, and in particular, antibiotic compounds which bind to bone and are useful in the treatment of bone infections. In work leading to the present disclosure, the inventors conjugated a cationic steroid antimicrobial (CSA) to a bone-binding moiety via a linker. The bone-binding moiety targets the CSA moiety to the bone, thereby increasing its concentration at the site of a bone infection for both treatment of existing infection and prophylaxis against subsequent infections or in cases of increased risk of infection.

Disclosed herein are compounds having the following Formula (I), or a pharmaceutically acceptable salt form thereof:

B-L-C (I)

wherein: B is a bone-binding moiety; L is a linker; and C is a cationic steroid antimicrobial (CSA) moiety.

In some embodiments, the CSA is selected from the group consisting of CSA-8, CSA-13, CSA-44, CSA-90, CSA-91, CSA-124, CSA-131, CSA-133, CSA-138, CSA-142, CSA-144, CSA-190, CSA-191, and CSA-192. In some embodiments, the CSA is CSA-90.

In some embodiments, the bone-binding moiety is a bisphosphonate, such as a bisphosphonate selected from the group consisting of etidronate, clodronate, tiludronate, pamidronate, medronate, etidronate, neridronate, olpadronate, alendronate, ibandronate, aminomethylene diphosphonate, risedronate and zoledronate. In some embodiments, the bisphosphonate is selected from the group consisting of alendronate, pamidronate and neridronate. In some embodiments, the bisphosphonate is alendronate.

In some embodiments, the linker is hydrophilic. In some embodiments, the linker has a molecular weight of less than about 2 kDa. In some embodiments, the linker comprises polyethylene glycol (PEG), such as where the linker has a structure of Formula (II):

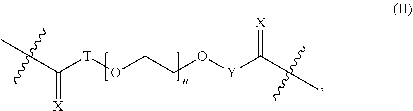

where,
X is independently selected from O and S;
T is absent or is an alkanediyl group having between 1 and 15 carbon atoms;
Y is absent or is an alkanediyl group having from 1 to 15 carbon atoms;
n is an integer from 1 to 30, and
the squiggly lines represent points of attachment to the CSA and bone-binding moieties.
   In some embodiments, X is O. In some embodiments, T is an alkanediyl group having from 1 to 15 carbon atoms. In some embodiments, Y is an alkanediyl group having from 1 to 15 carbon atoms. In some embodiments, T is an alkanediyl group having from 1 to 6 carbon atoms. In some embodiments, Y is an alkanediyl group having from 1 to 6 carbon atoms. In some embodiments, n is an integer from 10 to 20.

In some embodiments, the compound has a structure of Formula (III):

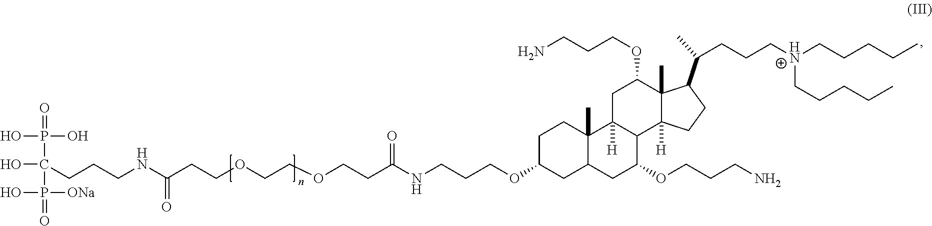

where n is from 1 to 50. In some embodiments, n is from 1 to 30. In some embodiments, the compound has a molecular weight of from about 1.5 kDa to about 2.5 kDa.

In some embodiments, the compound has a structure of Formula (IV):

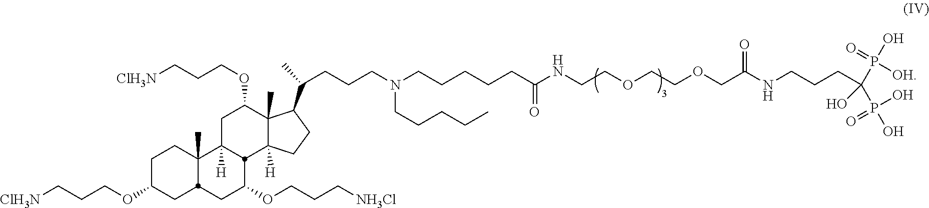

Also provided are pharmaceutical compositions comprising a compound disclosed herein and a pharmaceutically acceptable carrier. In some embodiments, the composition is suitable for systemic administration. In some embodiments, the composition is suitable for oral administration or parenteral administration. In some embodiments, the composition is suitable for intravenous administration.

Also provided are methods of treating an infection of a bone in a subject, comprising administering to the subject a compound or a pharmaceutical composition disclosed herein. In some embodiments, the infection is a bacterial infection, such as a Staphylococcus aureus infection, a Staphylococcus epidermidis infection, or a Pseudomonas aeruginosa infection. In some embodiments, the bone comprises a fracture.

Also provided are methods of treating osteomyelitis in a subject, comprising administering to the subject a compound or a pharmaceutical composition disclosed herein. In some embodiments, the osteomyelitis is associated with a Staphylococcus aureus infection, a Staphylococcus epidermidis infection, or a Pseudomonas aeruginosa infection.

Also provided are methods of promoting bone formation in a subject, comprising administering to the subject a compound or pharmaceutical composition disclosed herein. In some embodiments, the subject suffers from a bone disorder selected from the group consisting of a bone fracture, spinal cord injury, spinal disc degeneration, Paget's disease, bone cancer, metastatic bone cancer, and osteoporosis. In some embodiments, a bone of the subject is infected with one or more species of bacteria, such as one or more of Staphylococcus aureus, Staphylococcus epidermidis, or Pseudomonas aeruginosa.

In some embodiments, the compound or the pharmaceutical composition is administered systemically to the subject. In some embodiments, the compound or the pharmaceutical composition is administered orally or parenterally to the subject. In some embodiments, the compound or the pharmaceutical composition is administered intravenously to the subject. In some embodiments, the subject is a mammal. In some embodiments, the subject is a human.

Also provided is the use of a compound or pharmaceutical composition disclosed herein in the manufacture of a medicament for the treatment of an infection of a bone in a subject.

Also provided is the use of a compound or pharmaceutical composition disclosed herein in the manufacture of a medicament for the treatment of osteomyelitis in a subject.

Also provided is the use of a compound or pharmaceutical composition disclosed herein in the manufacture of a medicament for promoting bone formation in a subject.

Also provided is the use of a compound or pharmaceutical composition disclosed herein in the manufacture of a medicament for the treatment of bone cancer or metastatic bone cancer in a subject.

### BRIEF DESCRIPTION OF DRAWINGS

FIG. 1A-C. HPLC profiles of BBA-1 (FIG. 1A), pure NHS-PEG-COOH linker (FIG. 1B), and pure CSA-90 (FIG. 1C).

FIG. 2A-D. FT-IR spectra of alendronate (FIG. 2A), CSA-90 (FIG. 2B), NHS-PEG-COOH linker (FIG. 2C), and BBA-1 (FIG. 2D).

FIG. 3A. ¹H NMR spectra of BBA-1, NHS-PEG-COOH linker, CSA-90, and alendronate.

FIG. 3B. ³¹P NMR spectra of BBA-1 and alendronate.

FIG. 4A-C. FIG. 4A shows a Kirby Bauer assay against S. aureus and MRSA (results are from a single experiment containing triplicates of each sample). FIG. 4B shows representative image of zone of inhibition (Kirby Bauer assay against S. aureus and MRSA). FIG. 4C shows MIC and MBC of CSA-90 and BBA-1.

FIG. 5A-B. HA binding assays.

FIG. 6. Alkaline phosphatase activity assay (p-nitrophenyl phosphate) testing the pro-osteogenic effects of BBA-1 on cultured osteoblasts.

FIG. 7. Protein prenylation assay showing a lack of effect on the mevalonate pathway by BBA-1 and CSA-90.

FIG. 8. Data from toxicity study involving BBA-1 administered to mice showing infection relating to swab assay of soft tissue.

FIG. 9. Data from the toxicity study involving BBA-1 administered to mice showing infection relating to pin assay.

### DETAILED DESCRIPTION

### Definitions

The articles “a” and “an” are used herein to refer to one or to more than one (i.e. to at least one) of the grammatical object of the article.

The term “about” is understood to refer to a range of ±10%, such as ±5%, or ±1%, or ±0.1%.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and preferably together in the same formulation.

The terms “comprise”, “comprises”, “comprised” or “comprising”, “including” or “having” and the like in the present specification and claims are used in an inclusive sense, i.e., to specify the presence of the stated features but not preclude the presence of additional or further features.

The term “pharmaceutically acceptable” as used herein refers to substances that do not cause substantial adverse allergic or immunological reactions when administered to a subject. A “pharmaceutically acceptable carrier” includes, but is not limited to, solvents, coatings, dispersion agents, wetting agents, isotonic and absorption delaying agents, and disintegrants.

“Prevention” includes reduction of risk, incidence and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

The term “alkanediyl” is understood to refer to a bivalent saturated branched or straight chain hydrocarbon group conforming to the formula CₙH₂ₙ.

### Cationic Steroid Antimicrobial Moieties

Cationic steroid antimicrobials (CSAs), or ceragenins, are synthetic compounds designed to mimic the activities of endogenous antibacterial peptides. They are typically cationic and have a broad spectrum of antimicrobial activity including activity against bacteria, fungi and viruses, as well as anti-inflammatory and immunomodulatory activity. More than 100 CSAs have been synthesised.

CSAs like CSA-13, CSA-90 and CSA-131 have also been reported to promote or enhance osteogenesis (U.S. Pat. No. 9,694,019; Schindeler et al. J Bone Joint Surg Am. 2015. 97(4): 302-309). Bone is a dynamic tissue and its homeostasis represents a balance between bone formation and bone resorption. In bone formation, adult stem cells differentiate into bone progenitor cells (i.e., osteoprogenitor cells) that have the ability to mature into osteoblasts, osteocytes, and form mature bone and mineralized matrix. In bone resorption, osteoclasts (cells that resorb bone tissue) dissolve the mineralized matrix and create cavities on the bone surface. Despite the capacity for bone tissue to rejuvenate itself, repairing non-union bone fractures and regenerating bone defects remains a major challenge. Indeed, bone is now second only to blood as the most transplanted tissue. In this context, the compounds described herein may be useful for promoting bone formation and/or treating bone disorders.

CSA-90 is a small synthetic peptidomimetic compound based on endogenous cationic antibacterial peptides such as the human cathelicidin LL-37. LL-37 is found in the airway mucus and is thought to play an important role in controlling bacterial growth in the lung. The steroid-like structure of CSA-90 enables it to disrupt cell membranes and therefore confer a broad activity against Gram-positive and Gram-negative bacteria, including vancomycin- and methicillin-resistant strains.

The compounds described herein comprise a CSA moiety conjugated to a bone-binding moiety via a linker. The CSA moiety may be any suitable CSA such as CSA-8, CSA-13, CSA-44, CSA-90, CSA-91, CSA-124, CSA-131, CSA-133, CSA-138, CSA-142, CSA-144, CSA-190, CSA-191 or CSA-192. In some examples, the CSA is selected from the group consisting of:

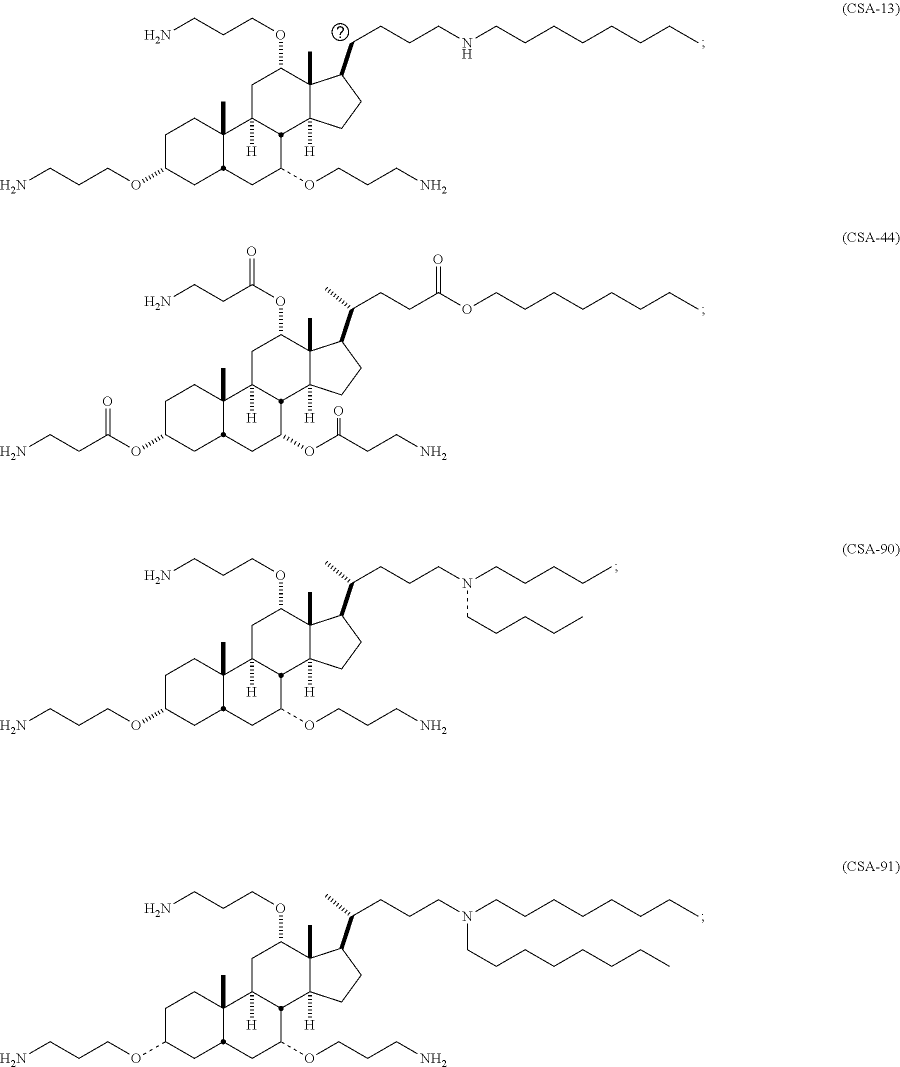

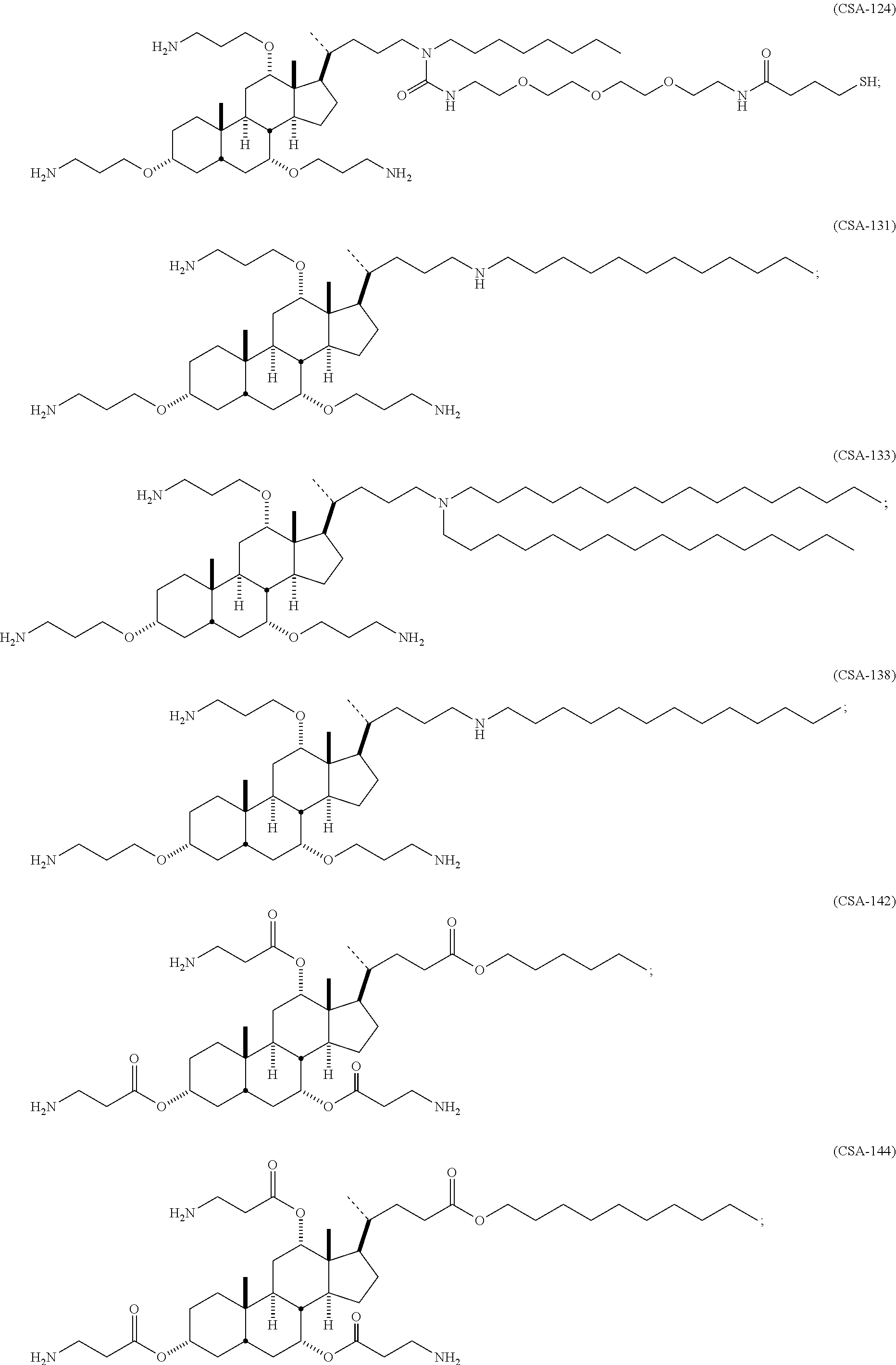

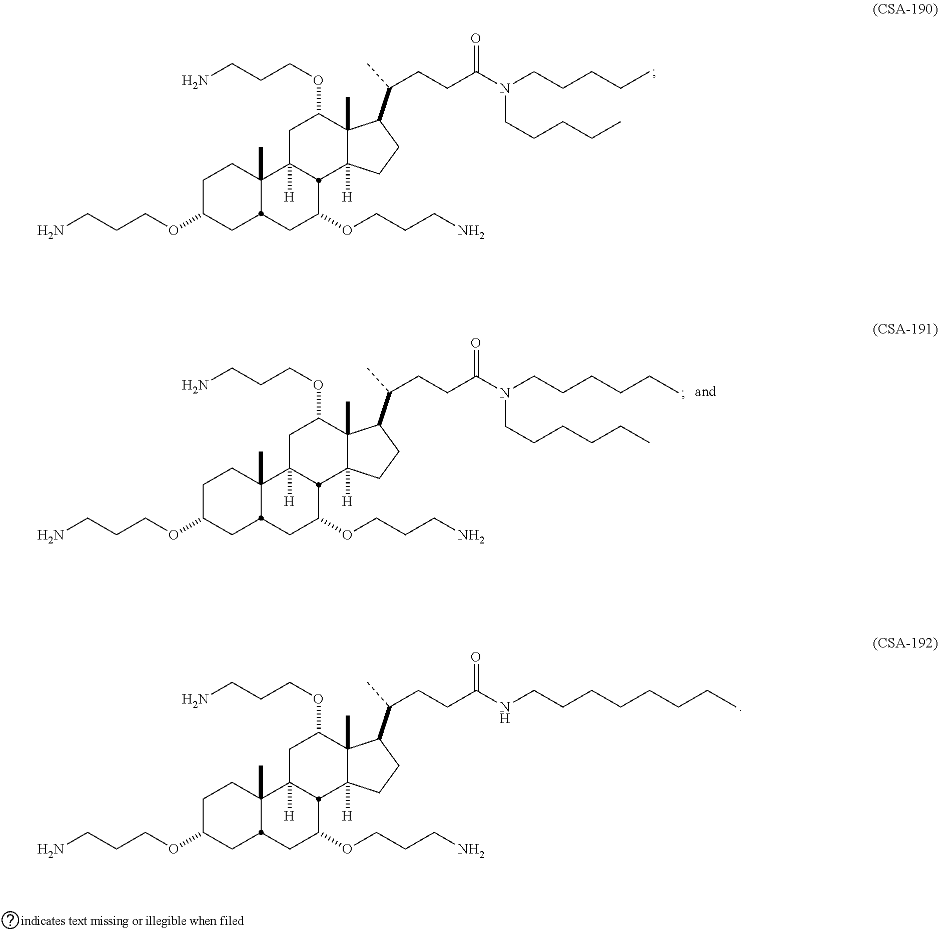

In some embodiments, the CSA moiety is CSA-13, CSA-90, or CSA-131. In certain examples, the CSA moiety is CSA-90.

The CSA moiety may be attached to the linker via an amino group of a CSA moiety or conjugated to the alkyl side of a CSA molecule.

Exemplary CSAs and methods for their manufacture are described in U.S. Pat. Nos. 6,350,738, 6,486,148, 6,767,904, 7,598,234, 7,754,705, 8,691,252, 8,975,310, 9,434,759, 9,527,883, 9,943,614, 10,155,788, 10,227,376, 10,370,403, and 10,626,139, U.S. Pat. Pub. Nos. 2016/0311850 and 2017/0210776, and U.S. Prov. Pat. App. Nos. 63/025,255 and 63/028,249, which are incorporated herein by reference.

### Bone-Binding Moiety

A bone-binding moiety as described herein is a chemical group that binds to bone, thereby targeting the compounds of the present disclosure to the bone. The bone-binding capability of a particular moiety can be assayed in a number of different ways. For example, bone binding can be assayed by binding to hydroxyapatite (HA), a mineral that is the main inorganic constituent of bone. A hydroxyapatite (HA) affinity assay may be performed by incubating a compound conjugated to the moiety in: i) water; and ii) water comprising HA. If the moiety is bone-binding, the amount of compound detected in the aqueous phase of the HA solution is expected to decrease as the compound will bind or absorb to the HA surface (See, e.g., Example 4). As a control, the compound lacking the bone-binding moiety can be assayed in the same way. Those skilled in the art will be familiar with other methods by which the bone-binding capability of a particular moiety may be assessed.

Suitable bone-binding moieties for use in the present disclosure may include, for example, a polyhydoxy-containing moiety, tetracycline derivative, acidic amino acid or peptide, hydoxylated hetercycleo, monophosphonate, bisphosphonate, antibody, or antigen-binding fragment. In some embodiments, the bone-binding moiety is a bisphosphonate.

Bisphosphonates generally comprise a phosphate-carbon-phosphate backbone and bind to hydroxyapatite (HA), the major mineral component found in bone, by coordination between the phosphate groups of the bisphosphonate and the calcium ions in the HA. The bisphosphonate may be attached to the linker via its terminal functional group (amine group in amino-bisphosphonates), geminal carbon or via either of its phosphate groups. In some embodiments, the linker is attached to the bisphosphonate via a terminal functional group attached to the geminal carbon of the bisphosphonate, such as for example an amino group or a hydroxy group, or other reactive group. Examples of bisphosphonate bone-binding moieties that may be used in accordance with the present disclosure may include etidronate, clodronate, tiludronate, pamidronate, medronate, etidronate, neridronate, olpadronate, alendronate, ibandronate, aminomethylene diphosphonate, risedronate and zoledronate. Preferably, the bone-binding moiety is alendronate.

### Linkers and Compound Variants

The linker described herein covalently attaches a CSA moiety to a bone-binding moiety as set forth in Formula (I):

B-L-C (I)

wherein B is a bone-binding moiety, L is a linker, and C is a CSA. It will be understood that Formula (I) is not directional and may equally be represented as C-L-B.

The linker may be small, consisting of a single covalent bond, or it may be larger moiety reaching 10 kDa or more. In some embodiments, the linker has a molecular weight of less than about 10 kDa, such as less than about 9 kDa, or less than about 8 kDa, or less than about 7 kDa, or less than about 6 kDa, or less than about 5 kDa, or less than about 4 kDa, or less than about 3 kDa, or less than about 2 kDa, such as about 1 kDa or less. In certain examples, the linker is from about 0.2 kDa to 10 kDa, such as about 0.3 kDa to 9 kDa, or about 0.4 kDa to 8 kDa, or about 0.5 kDa to 7 kDa, or about 0.5 kDa to 6 kDa, or about 0.5 kDa to 5 kDa, or about 0.5 kDa to 4 kDa, or about 0.5 kDa to 3 kDa, or about 0.5 kDa to 2 kDa, or about 0.5 kDa to 1.5 kDa.

The linker may include, for example, an ether, ester, thioester, phosphoester, amide, peptide (e.g., dipeptide), polypeptide, polysaccharide, hydrophobic linker (e.g., strait alkane, fatty acid, etc.), or any combination thereof. The linker may be a cleavable linker, such as a hydrolysable linker (e.g., carbamate linker) or cathepsin-sensitive linker, or non-cleavable linker. The linker is preferably stable in the blood stream for a sufficient period of time (e.g., more than 1 hour, such as more than 6 hours, or more than 12 hours, or more than 18 hours, or more than 24 hours) to enable the compound to reach the bone. The linker may be hydrophilic. In some embodiments, the linker is, or comprises, polyethylene glycol (PEG). The PEG linker may have a molecular weight as defined in the preceding paragraph.

In one embodiment the linker has a structure of Formula (II):

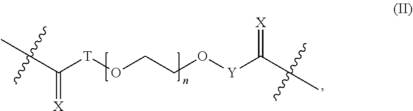

wherein:

X is independently selected from O and S;

T is absent or is an alkanediyl group having from 1 to 15 carbon atoms;

Y is absent or is an alkanediyl group having from 1 to 15 carbon atoms;

n is an integer between 1 and 30; and

the squiggly lines represent points of attachment to the CSA and bone-binding moieties.

T may be an alkanediyl group having from 1 to 12 carbon atoms, or 1 to 10 carbon atoms, or 1 to 8 carbon atoms, or 1 to 6 carbon atoms, or 1 to 5 carbon atoms, or 1 to 4 carbon atoms.

Y may be an alkanediyl group having from 1 to 12 carbon atoms, or 1 to 10 carbon atoms, or 1 to 8 carbon atoms, or 1 to 6 carbon atoms, or 1 to 5 carbon atoms, or 1 to 4 carbon atoms.

In some embodiments T and Y are —CH₂CH₂—.

n may be an integer from about 2 to about 25, or from about 2 to about 25, or from about 3 to about 25, or from about 4 to about 25, or from about 5 to about 25, or from about 6 to about 25, or from about 2 to about 20, or from about 2 to about 20, or from about 3 to about 20, or from about 4 to about 20, or from about 5 to about 20, or from about 6 to about 20, or from about 10 to about 30, or from about 10 to about 25, or from about 10 to about 20, or from about 15 to about 25, or from about 12 to about 20.

In some embodiments the linker may be attached to the CSA moiety via an amino group of the CSA. Likewise, the linker may be attached to the bone-binding moiety via an amino group of the bone-binding moiety.

The compound represented by Formula (I) may comprise other features, such as further conjugates or moieties. For example, the compound may comprise a second antibiotic moiety in addition to the CSA. In such examples, the CSA may be directly conjugated to the second antibiotic moiety, for example, in the form B-L-C-A₂, wherein A₂ is a second antibiotic moiety. Alternatively, the CSA and the second antibiotic moiety may be attached at opposite ends of the compound, for example, in the form A₂-B-L-C. Suitable second antibiotics may include ciprofloxacin, gemcitabine, paclitaxel, cytarabine, rifalazil, norfloxacin, enoxacin, gatifloxacin, moxifloxacin, a fluoroquinolone ester, a benzoxazinorifamycine, aminoglycoside, polyene, nitroimidazole, rifamycin, bacitracin, a beta-lactam, cephalosporin, chloramphenicol, a glycopeptide, a macrolide, a lincosamide, penicillin, a quinolones, rifampicin, tetracycline, trimethoprim a sulfonamide, amoxicillin, augmentin, amoxicillin, ampicillin, azlocillin, flucloxacillin, mezlocillin, methicillin, cephalexin, cefazedone, cefuroxime, loracarbef, cemetazole, cefotetan, cefoxitin, ciprofloxacin, levaquin, floxacin, doxycycline, minocycline, gentamycin, amikacin, tobramycin, clarithromycin, azithromycin, erythromycin, daptomycin, neomycin, kanamycin, streptomycin, nisin, epidermin, gallidennin, cinnamycin, duramycin, lacticin 481, amoxicillin, amoxicillin/clavulanic acid, metronidazole, clindamycine, chlortetracycline, dcmeclocycline, oxytetracycline, amikacin, netilmicin, cefadroxil, cefazolin, cephalexin, cephalothin, cephapirin, cephradine, cefaclor, cefamandole, cefametazole, cefonicid, cefotetan, cefoxitine, cefpodoxime, cefprozil, cefuroxime, cefdinir, cefixime, cefoperazone, cefotaxime, ceftazidime, ceftibuten, ceftizoxime, ceftriaxone, cefepime, azithromycin, claforan, clarithromycin, dirithromycin, erythromycin, lincomycin, troleandomycin, bacampicillin, carbenicillin, cloxacillin, dicloxacillin, meticillin, mezlocillin, nafcillin, oxacillin, piperacillin, ticarcillin, cinoxacin, ciprofloxacin, enoxacin, grepafloxacin, levofloxacin, lomefloxacin, nalidixic acid, norfloxacin, ofloxacin, sparfloxacin, sulfisoxazole, sulfacytine, sulfadiazine, sulfamethoxazole, sulfisoxazole, dapson, aztreonam, capreomycin, clofazimine, colistimethate, colistin, cycloserine, fosfomycin, furazolidone, methenamine, nitrofurantoin, pentamidine, rifabutin, spectinomycin, tigecycline, trimethoprim, trimetrexate glucuronate, vancomycin, chlorhexidine, carbapenem or ertapenem.

The compound may comprise more than one bone-binding moiety. For example, the bone-binding moieties may be directly attached to each other, such as in the form B₁-B₂-L-C, or they may be attached at opposite ends of the compound, such as in the form B₁-L-C—B₂, wherein B₁ and B₂ are the same or different bone-binding moieties. Further combinations and additions of antibiotic and bone-binding moieties are envisaged by the present disclosure and will be clear to a person skilled in the art.

In certain examples, the present disclosure provides a compound having the structure set forth as Formula (III) (BBA-1):

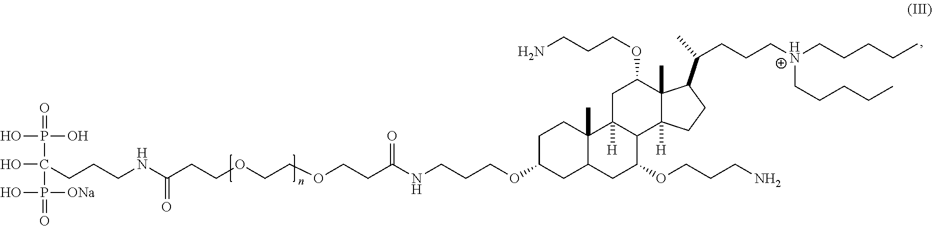

wherein n is from 1 to 100. In certain examples, n is from 1 to 95, or from 1 to 90, or from 1 to 85, or from 1 to 80, or from 1 to 75, or from 1 to 70, or from 1 to 65, or from 1 to 60, or from 1 to 55, or from 1 to 50, or from 1 to 45, or from 1 to 40, or from 1 to 35, or from 1 to 30, or from 5 to 30, or from 5 to 25, or from 5 to 20, or from 10 to 20. The compound may have a molecular weight of from about 1 kDa to 10 kDa, such as from about 1 kDa to 9 kDa, or from about 1 kDa to 8 kDa, or from about 1 kDa to 7 kDa, or from about 1 kDa to 6 kDa, or from about 1 kDa to 5 kDa, or from about 1 kDa to 4 kDa, or from about 1 kDa to 3 kDa, or from about 1.5 kDa to 3 kDa, or from about 1.5 kDa to 2.5 kDa, or from about 1.75 kDa to 2.25 kDa, such as about 2 kDa.

In certain examples, the present disclosure provides a compound having the structure set fourth as Formula (IV) (BBA-2):

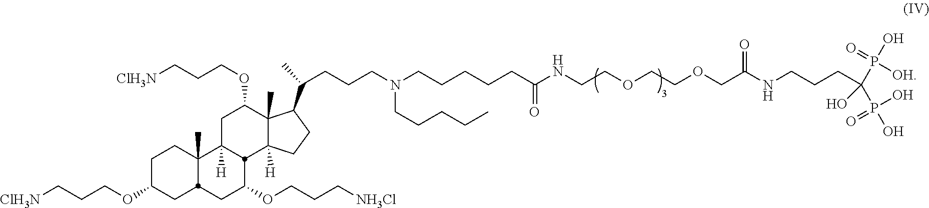

The compounds of Formula (I) may, for example, be prepared by reacting an appropriately functionalised linker with a CSA and a bone-binding moiety. One such example is shown below in Scheme 1.

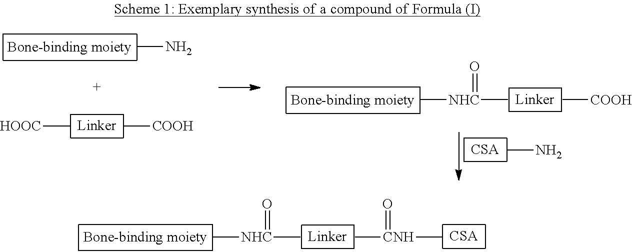

In scheme 1, a bone-binding moiety having an amino group is reacted with a linker functionalized with two terminal carboxylic acid groups (in which one carboxylic acid group may be activated) so as to provide an intermediate in which the bone-binding moiety is attached to the linker via an amide bond. The carboxylic acid present in the intermediate is then reacted with an amino group of a CSA to generate another amide bond and thereby provide the compound of Formula (I). Those skilled in the art will be familiar with alternative synthetic methodologies that may be used to prepare compounds of Formula I.

### Methods and Compositions

The compounds described herein target bone by way of their bone-binding moiety and may be used to treat an infection of a bone. The infection may be a bacterial infection such as a Gram-positive bacterial infection or a Gram-negative bacterial infection. In some examples, the bacterial infection is a staphylococcus or pseudomonas infection, such as a Staphylococcus aureus, Staphylococcus epidermidis, or Pseudomonas aeruginosa infection, such as a methicillin-resistant Staphylococcus aureus, methicillin-resistant Staphylococcus epidermidis, or tobramycin-resistant Pseudomonas aeruginosa infection. In some examples, the bacterial infection is a vancomycin-resistant Staphylococcus aureus. Although Staphylococcus epidermidis is sometimes considered a less virulent pathogen than Staphylococcus aureus, reports indicate that Staphylococcus epidermidis strains may be acquiring more invasive properties and are just as effective at forming biofilms, particularly on orthopedic implants (Gill et al. J Bacteriol. 2005. 187: 2426-2438). In some examples, the bacterial infection is an Aggregatibacter actinomycetemcomitans infection. Aggregatibacter actinomycetemcomitans is commonly observed in cases of jawbone osteomyelitis.

In other embodiments, the bone infection is a fungal infection, such as an infection with a Candida species.

In some embodiments, the infections result from an orthopedic implant, osteomyelitis, or surgical site infection. In some embodiments, the disclosed compounds are useful for the treatment of infections and, in other embodiments, the disclosed compounds are useful for the prophylaxis of infection in subjects susceptible to, or at risk of, bone infections.

CSAs like CSA-13, CSA-90, and CSA-131 have also been reported to promote or enhance osteogenesis (U.S. Pat. No. 9,694,019; Schindeler et al. J Bone Joint Surg Am. 2015. 97(4): 302-309). In that regard, the compounds described herein may be used to promote bone formation in a subject. Bone disorders that may be treated in accordance with the present disclosure include, for example, a bone fracture, a spinal cord injury, spinal disc degeneration, Paget's disease, bone cancer, and osteoporosis. Bone fractures that may be treated using the compositions and methods of the present disclosure include non-union fractures, simple fractures, greenstick fractures, compound fractures, comminuted (multi-fragmentary) fractures, impacted fractures, complicated fractures, hairline fractures, compression fractures, fatigue fractures, and/or pathological fractures. Examples of bone fractures that may be advantageously treated by the methods described herein include, but are not limited to, fractures of the spine, leg, and arm. A further example of a fracture that may be advantageously treated in accordance with the present disclosure is a vertebral compression fracture. Such fracture occurs when one or more of the bones of the vertebral column fractures or collapses, typically when the vertebrae are already weakened for instance as a result of ageing or a disease that weakens bone, such as osteoporosis, Paget's disease, or bone cancer. In some examples, the bone diseases or conditions that may be treated in accordance with the present disclosure include bone resorption, osteoarthritis, osteoporosis, osteomalacia, osteitis fibrosa cystica, osteochondritis dissecans, osteomalacia, osteomyelitis, osteoblastogenesis, osteopenia, osteonecrosis, and porotic hyperostosis.

The compositions and methods of the present disclosure may be used to treat a subject suffering from an imbalance in bone formation and resorption. Imbalance of bone formation and resorption usually causes loss of bone mass and can lead to bone related diseases, such as osteoporosis, rickets, and osteomalacia. These bone diseases are associated with increased risk of bone fractures, increased severity of fractures and protracted time periods for healing. Additionally, with age or injury the incidence of disc degenerative disease or deformity of the spine is increased, leading to spondylolisthesis.

Dosages may vary with the type and severity of the condition to be treated and may include single or multiple dosses. Specific dosage regimens may be adjusted over time according to the individual need and the professional judgment of the practitioner administering the compound. When administered to a human subject, the dosage regimen may vary depending on a variety of factors including the type and severity of infection or condition, the age, sex, weight or medical condition of the subject and the route of administration. In that regard, the precise amount of the compound that is to be administered may depend on the judgement of the practitioner. In some embodiments, the subject can be a mammal. In some embodiments, the subject is a human, a companion animal (e.g., dog, cat, ferret, hamster, gerbil, etc.), a livestock animal (e.g., cattle, pig, horse, poultry, etc.), or any other mammal in need of treatment.

The compounds described herein may be administered over a period of hours, days, weeks, or months, depending on several factors, including the severity of the infection or condition being treated, whether a recurrence is considered likely. The administration may be via infusion over a period of hours, days, weeks, months, etc. Alternatively, the administration may be intermittent, e.g., once per day over a period of days, once per hour over a period of hours, or any other such schedule as deemed suitable. In some embodiments, the composition of the present disclosure is administered once daily for at least one week, for example, at least once daily for at least two weeks, or once daily for at least one month or longer. In other embodiments, the composition of the present disclosure is provided immediately prior, or subsequent, to exposure to an infectious agent, or upon onset of risk for a bone infection.

In some examples, the compound of the present disclosure is administered at an amount of from about 0.01 mg/kg to 1000 mg/kg of body weight. For example, the compound of the present disclosure may be administered at an amount of from about 0.1 mg/kg to 900 mg/kg, or from about 0.5 mg/kg to 800 mg/kg, or from about 1 mg/kg to 750 mg/kg, or from about 1.5 mg/kg to 700 mg/kg, or from about 2 mg/kg to 600 mg/kg, or from about 2 mg/kg to 500 mg/kg, or from about 2.5 mg/kg to 450 mg/kg, or from about 3 mg/kg to 350 mg/kg, or from about 3.5 mg/kg to 250 mg/kg, or from about 4 mg/kg to 200 mg/kg, or from about 4 mg/kg to 100 mg/kg, or from about 4 mg/kg to 50 mg/kg, or from about 4 mg/kg to 20 mg/kg, or any dose bounded by these ranges. In some embodiments, the compound of the present disclosure is administered at about 2 mg/kg, about 5 mg/kg, about 8 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg, or within any range bounded by the foregoing dosage amounts. In some examples, the antibiotic compound is administered systemically.

It will be understood that the targeting conferred by the bone-binding moiety may reduce the dosing that is required to provide an effective amount of the antibiotic (e.g., CSA) at the site of infection compared to the dosing that would be required in the absence of a bone-binding moiety. It will also be understood that higher doses of the bone-binding compound may be tolerated by a patient compared to the doses that may be tolerated of an antibiotic compound lacking bone-binding capabilities, as the bone-binding antibiotic compound is directed to the tissue of interest rather than travelling non-specifically to other locations in the body.

In some examples, the present disclosure provides an oral dose of from about 0.01 mg to 4000 mg of the active ingredient, such as from about 0.05 mg to 3500 mg, or from about 0.1 mg to 3000 mg, from about 0.5 mg to 2500 mg, from about 0.75 mg to 2000 mg, or from about 1 mg to 1750 mg, from about 1.25 mg to 1500 mg, or from about 1.5 mg to 1250 mg, or from about 2 mg to 1000 mg, or from about 5 mg to 900 mg, from about 7.5 mg to 800 mg, or from about 10 mg to 700 mg, or from about 15 mg to 600 mg, or from about 20 mg to 550 mg, or from about 25 mg to 500 mg, or from about 30 mg to 500 mg, or from about 35 mg to 450 mg, or from about 40 mg to 450 mg, or from about 45 mg to 450 mg, or from about 50 mg to 400 mg.

Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, transcutaneous, intradermal, or intramedullary delivery (e.g., injection), as well as intrathecal, direct intraventricular, intravenous, intraperitoneal, intranasal, pulmonary, transdermal, or intraocular delivery (e.g., injection).

Components may be formulated to permit release over a prolonged period of time. A release system can include a matrix of a biodegradable material or a material which releases the incorporated components by diffusion. The components can be homogeneously or heterogeneously distributed within the release system. A variety of release systems may be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). The release system material can be selected so that components having different molecular weights are released by diffusion or through degradation of the material. Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives, such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof. Poly(lactide-co-glycolide) microspheres or nanospheres can be used.

The compounds of the present disclosure may be administered in combination with additional antibiotic agents. Suitable antibiotic agents may include, for example, ciprofloxacin, gemcitabine, tryptophan, paclitaxel, cytarabine, rifalazil, norfloxacin, enoxacin, gatifloxacin, moxifloxacin, a fluoroquinolone ester, a benzoxazinorifamycine, aminoglycoside, polyene, nitroimidazole, rifamycin, bacitracin, a beta-lactam, cephalosporin, chloramphenicol, a glycopeptide, a macrolide, a lincosamide, penicillin, a quinolones, rifampicin, tetracycline, trimethoprim a sulfonamide, amoxicillin, augmentin, amoxicillin, ampicillin, azlocillin, flucloxacillin, mezlocillin, methicillin, cephalexin, cefazedone, cefuroxime, loracarbef, cemetazole, cefotetan, cefoxitin, ciprofloxacin, levaquin, floxacin, doxycycline, minocycline, gentamycin, amikacin, tobramycin, clarithromycin, azithromycin, erythromycin, daptomycin, neomycin, kanamycin, streptomycin, nisin, epidermin, gallidennin, cinnamycin, duramycin, lacticin 481, amoxicillin, amoxicillin/clavulanic acid, metronidazole, clindamycine, chlortetracycline, dcmeclocycline, oxytetracycline, amikacin, netilmicin, cefadroxil, cefazolin, cephalexin, cephalothin, cephapirin, cephradine, cefaclor, cefamandole, cefametazole, cefonicid, cefotetan, cefoxitine, cefpodoxime, cefprozil, cefuroxime, cefdinir, cefixime, cefoperazone, cefotaxime, ceftazidime, ceftibuten, ceftizoxime, ceftriaxone, cefepime, azithromycin, claforan, clarithromycin, dirithromycin, erythromycin, lincomycin, troleandomycin, bacampicillin, carbenicillin, cloxacillin, dicloxacillin, meticillin, mezlocillin, nafcillin, oxacillin, piperacillin, ticarcillin, cinoxacin, ciprofloxacin, enoxacin, grepafloxacin, levofloxacin, lomefloxacin, nalidixic acid, norfloxacin, ofloxacin, sparfloxacin, sulfisoxazole, sulfacytine, sulfadiazine, sulfamethoxazole, sulfisoxazole, dapson, aztreonam, capreomycin, clofazimine, colistimethate, colistin, cycloserine, fosfomycin, furazolidone, methenamine, nitrofurantoin, pentamidine, rifabutin, spectinomycin, tigecycline, trimethoprim, trimetrexate glucuronate, vancomycin, chlorhexidine, carbapenem, or ertapenem.

The compounds of the present disclosure may be administered in combination with additional compounds that are useful for promoting bone formation or decreasing bone resorption. For example, suitable compounds may include risedronate (Actonel), Ibandronate (Boniva), or zoledronic acid (Reclast or Aclasta). Alternatively, or in addition, the other compound may be a corticosteroid, e.g., prednisone or cortisone. Alternatively, or in addition, the other compound may be denosumab (Prolia). Alternatively, or in addition, the other compound may be strontium ranelate (Protos). Alternatively, or in addition, the other compound may be a selective estrogen receptor modulator (SERMS), such as raloxifene (Evista). Alternatively, or in addition, the other compound may be a drug used in hormone replacement therapy (HRT), such as estrogen or progesterone. Alternatively, or in addition, the other compound may be teriparatide (Forteo). Alternatively, or in addition, the other compound may be a non-steroidal anti-inflammatory agent or analgesic. For example, a suitable non-steroidal anti-inflammatory agent may be ibuprofen, naproxen, aspirin, or a COX-1 and/or COX-2 inhibitor selected from ketoprofen, indomethacin (Indocin or Tivorbex), and fenoprofen (Nalfon).

The compound of the present disclosure may be administered to a subject in association with a scaffold. The scaffold material may be as described in U.S. Pat. Nos. 5,681,872; 5,914,356; 5,939,039; 6,325,987; 6,383,519; 6,521,246; 6,736,799; 6,800,245; 6,969,501; 6,991,803; 7,052,517; 7,189,263; 7,534,451; 8,303,967; 8,460,686; or 8,647,614, which are incorporated by reference. Other suitable scaffold materials may include VITOSS®, CORTOSS®, biopolymers, bone, decellularized bone, extracellular matrix or components thereof, fibronectin, laminin collagen, chitosan, alginate, calcium phosphate, calcium sulfate, poly(alpha-hydroxy acids) such as poly(lactic-co-glycolic acid) and polyglycolic acid, CUPE polymer, polyethylene glycol, or any combinations thereof. The scaffold material may be porous. The scaffold material may be a natural material, synthetic material, or a combination thereof. The scaffold material may be biocompatible, nontoxic and/or non-inflammatory. The scaffold material may support cell attachment, cell proliferation, extracellular and/or bone matrix production, and/or cell conversion. The scaffold material may be biodegradable. The scaffold material may be sterilized. Other scaffold materials and attributes will be appreciated by those of skill in the art.

The compounds of the present disclosure may be administered by a variety of routes. In some embodiments, the compound is administered systemically such as by direct delivery into the bloodstream of a subject. In certain embodiments, the compound is delivered parenterally. Exemplary routes of parenteral administration include, but are not limited to, intravascular, intracapsular, intraorbital, intracardiac, intradermal, transtacheal, intraperitoneal, intraventricular, intracerebroventricular, intrathecal, subcutaneous, subcuticular, subcapsular, subarachnoid, intraspinal, epidural, intrasternal, intracranial, intramuscular, intraarticular, intra-arterial, intranodal, pulmonary, intranasal, transdermal, and intravenous. In certain examples, the compound is administered intraperitoneally or intravenously.

Alternatively, the compounds may be formulated using pharmaceutically acceptable carriers well known in the art into dosage forms suitable for oral administration such as tablets, pills, capsules, liquids, gels, syrups, slurries, suspensions, and the like. Suitable carriers may be selected from malt, gelatin, talc, calcium sulphate, vegetable oils, synthetic oils, polyols, alginic acid, phosphate buffered solutions, emulsifiers, isotonic saline, and pyrogen-free water. In certain examples, the compound is administered intranasally or by inhalation (for example, in the form of an aerosol spray presentation from a pressurized container, pump, spray or nebulizer with the use of a suitable propellant, such as dichlorodifluoromethane, trichlorofluoro-methane, dichlorotetra-fluoro-ethane, a hydrofluoroalkane such as 1,1,1,2-tetrafluoroethane (HFA 134A3 or 1,1,1,2,3,3,3-heptafluoropropane (HFA 227EA3), carbon dioxide, or other suitable gas). or as solid micronized powder delivered with dry powder inhaler

### EXAMPLES

### Example 1: Synthesis

A compound referred to as Bone-Binding Antibiotic-1 (BBA-1) was synthesized in a two-step reaction. In the first step, a NHS-PEG-COOH linker (1 kDa, 33 mg) was dissolved in 1 mL of Milli-Q water. Separately, alendronate (ALN) sodium (10.5 mg) was weighed and dissolved in 1 mL of Milli-Q water. Alendronate solution was added dropwise into the solution of NHS-PEG-COOH under continuous stirring. pH of the reaction was monitored and adjusted to pH 7 using NaOH solution. The reaction mixture was stirred overnight at room temperature. Following the overnight reaction, unconjugated alendronate was precipitated by adding 3.5 mL of absolute ethanol and removed by filtration using a 0.45 micron nylon filter to yield ALN-PEG-COOH. In the second step, 1-ethyl-3-(3-dimethylaminopropyl) carbodiimide (hydrochloride EDC.HCL 30 mg) and CSA-90 (25.5 mg) were added to the filtered solution comprising ALN-PEG-COOH. The reaction mixture was stirred overnight at room temperature to yield the final conjugate product (ALN-PEG-CSA-90). The reaction solution was placed in a 1 kDa dialysis tube and dialyzed for 24 hours using water as the dialysis medium. After dialysis, the purified reaction product was freeze dried and used for identification and chemical characterization using HPLC, NMR, and FT-IR spectroscopy.

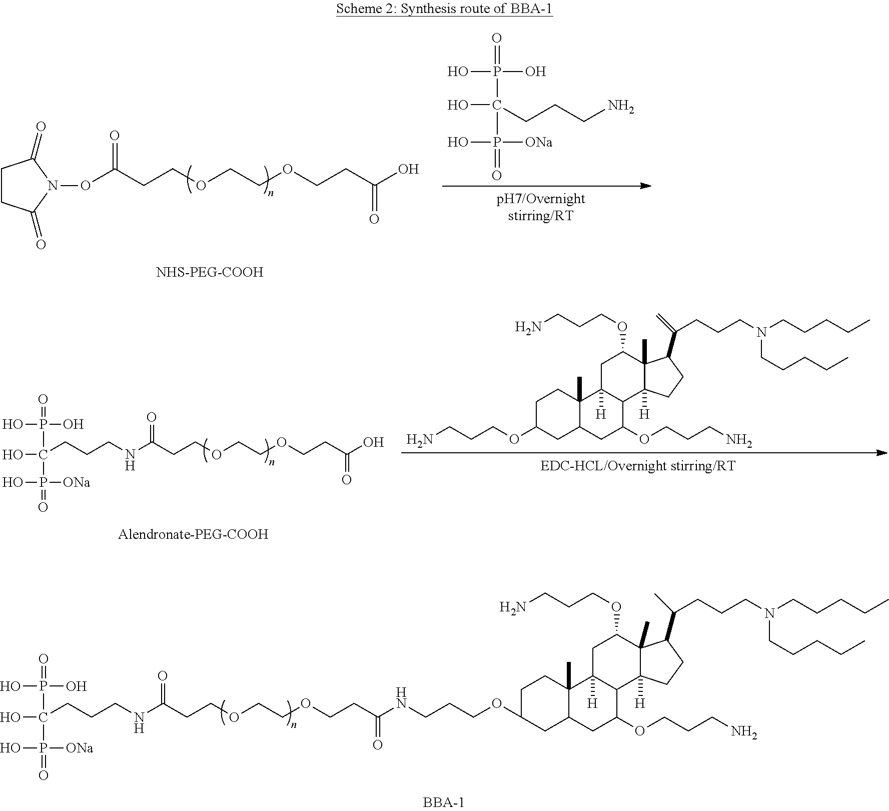

BBA-1 has a molecular weight of about 2070.12 g/mole and its structure is:

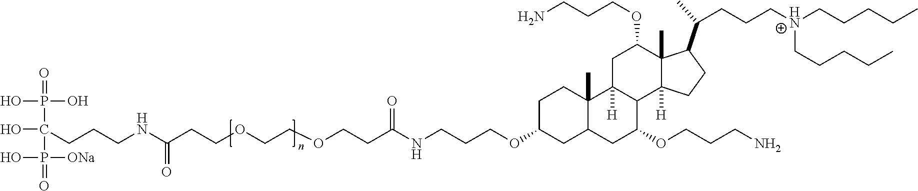

in which n is about 16.

### Example 2: Characterization

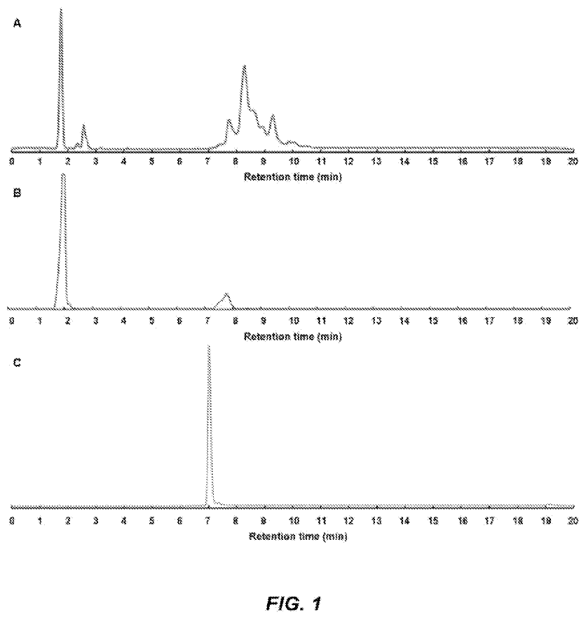

BBA-1 was characterized using high performance liquid chromatography (HPLC). Referring to FIG. 1-A, a broad peak at 8.2 min and 9.3 min was attributed to the possible conjugation of ALN-PEG-COOH linker to the two primary amino groups of CSA-90. A peak at 7.5 min was attributed to unreacted linker, while a peak at 2.5 min was due to the EDC.HLC carbodiimide. Corresponding elution and identification of pure NHS-PEG-COOH linker at time 5 mins and CSA-90 at 6.8 min using similar method is shown in FIG. 1-B and FIG. 1-C, respectively. The subtle or lack of a peak of CSA-90 at 6.8 min, and the significant reduction of the NHS-PEG-COOH peak at 7.5 min in the BBA-1 spectrum (FIG. 1-A) of the BBA-1 reaction mixture indicates the maximum conjugation efficiency of CSA-90 to the linker.

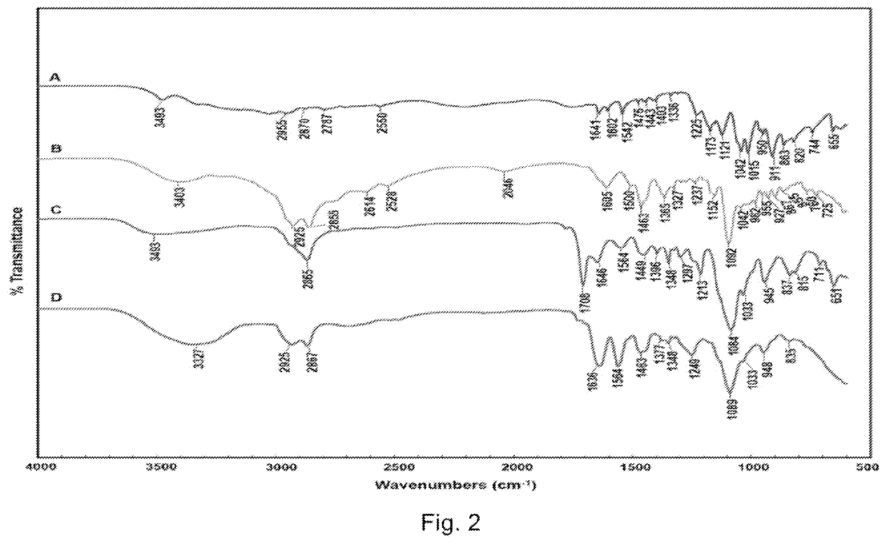

FIG. 2 shows FT-IR spectrums for alendronate (FIG. 2-A), CSA-90 (FIG. 2-B), NHS-PEG-COOH linker (FIG. 2-C) and BBA-1 (FIG. 2-D), respectively. The existence of CSA-90 in the spectrum of BBA-1 (FIG. 2-D) was confirmed by the identification of peaks at 2867 cm⁻¹ and 2925 cm⁻¹. In all spectra, the peaks around 1550-1560 cm⁻¹ and above 3300 cm⁻¹ correspond to N—H bending and stretching vibrations, respectively (FIG. 2-A to 2-D). After conjugation of CSA-90 and alendronate to a NHS-PEG-COOH linker, the stretching vibration of the C═O bond due to the formation of an amide linkage with CSA-90, and alendronate was recorded at 1636 cm⁻¹ in the spectrum of BBA-1 (FIG. 2-D).

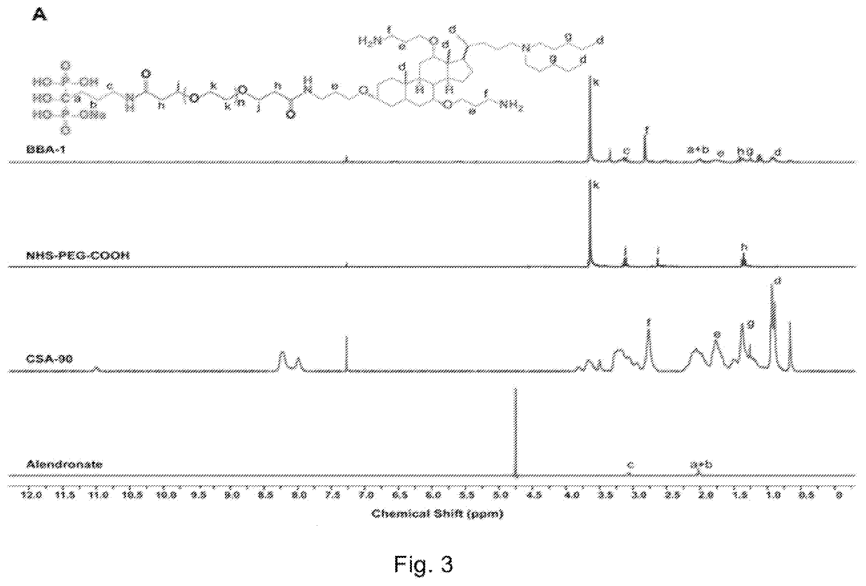

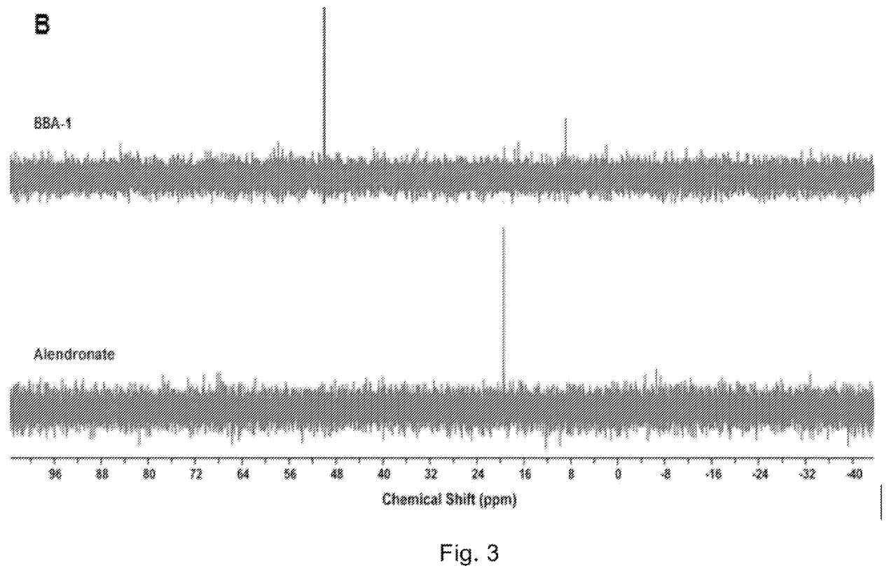

As described in Example 1, alendronate was conjugated to the NHS terminus of the NHS-PEG-COOH linker. In a second step, CSA-90 was conjugated to the COOH terminus of the linker using water soluble 1-ethyl-3-(3-dimethylaminopropyl)-carbodiimide (EDC.HCL). Excess EDC.HCL, CSA-90, and alendronate were removed by precipitation, filtration, and dialysis. FIG. 3-A shows the ¹H NMR spectrums for BBA-1, NHS-PEG-COOH, CSA-90, and alendronate sodium. The success of alendronate conjugation was confirmed by ¹H NMR and ³¹P NMR spectroscopy. As shown in the ¹H NMR spectrum for BBA-1 (FIG. 3-A), the corresponding peaks at 2.01 ppm and 3.14 ppm (a and b) were attributed to CH₂C and CH₂CH₂ from the alendronate, respectively, indicating successful alendronate conjugation. Disappearance of a peak characteristic to CH₂—CH₂ of the NHS from the NHS-PEG-COOH linker additionally confirmed the alendronate conjugation to NHS termini of the linker (FIG. 3A, BBA-1 and NHS-PEG-COOH spectra). Similarly, identification of characteristic peaks of CSA-90 at 0.93 ppm, 1.76 ppm, 2.82 ppm, and 1.25 ppm confirmed the conjugation of CSA-90 to the carboxylic termini of ALN-PEG-COOH. Typical protons due to ethylene oxide from the NHS-PEG-COOH linker were identified at 3.64 ppm (FIG. 3A, BBA-1). Additionally, alendronate conjugation was qualitatively confirmed by ³¹P NMR spectroscopy (FIG. 3-B). A corresponding peak of phosphorus (P) from alendronate at 19.42 ppm (FIG. 3B, Alendronate) was shifted to 50.06 ppm in the ³¹P NMR of BBA-1, (FIG. 3-B, BBA-1) which indicates a positive qualitative conjugation signal.

### Example 3: Antibacterial Assays

A modified Kirby-Bauer disc diffusion assay was performed to test the bactericidal activity of BBA-1 against common species of bacteria causing bone infection. Whatman filter paper discs N^{Ø}1 (6 mm) containing equimolar concentration of BBA-1 or CSA-90 to standard 200 μg gentamicin discs were prepared and compared. Briefly, 0.5 mL of Staphylococcus aureus and methicillin-resistant Staphylococcus aureus (MRSA) (diluted to OD₆₀₀ of 0.2=1.6×10⁸ bacteria/mL) was spread evenly on LB agar plates. Antibiotic discs were placed on the LB agar plates and the zone of bacterial growth inhibition around each antibiotic disc was measured after overnight incubation. Furthermore, minimum inhibitory concentration (MIC) and minimum bactericidal concentration (MBC) of BBA-1 was determined by standardized broth microdilution method. Briefly, 1×10⁵ CFU/mL of S. aureus and MRSA inoculum was incubated with serial concentrations of BBA-1 and CSA-90. The optical density/absorbance was recorded in triplicate at 595 nm after 24 hours of incubation at 37° C.

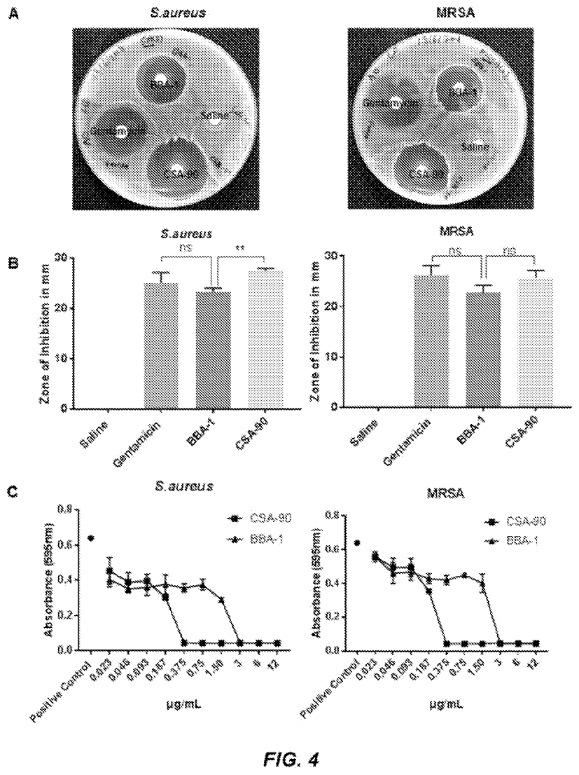

As shown in FIGS. 4-A and 4-B, BBA-1-treated discs significantly inhibited bacterial growth across all experiments. The antibacterial activity of BBA-1 at an equimolar concentration of CSA-90 and gentamicin was comparable against both strains of bacteria. The MIC and MBC concentration of BBA-1 against S. aureus and MRSA were 1.5 μg/mL and 3 μg/mL, respectively (FIG. 4-C).

### Example 4: Bone-Binding Assay

Hydroxyapatite (HA) binding affinity of BBA-1 and CSA-90 was determined semi-quantitatively by incubating each compound with and without HA in milli-Q water. BBA-1 (2.42 mg) was weighed and dissolved in 4 mL of milli-Q water. Separately, HA (20 mg) was weighed and suspended in 2 mL of BBA-1 solution, and the suspension was incubated at room temperature. To analyze the reduction in peak height due to HA binding, 100 μL of supernatant was withdrawn at predetermined time points and analyzed by HPLC. Similarly, bone binding ability of pure CSA-90 was tested by incubating CSA-90 with and without HA at similar concentrations.

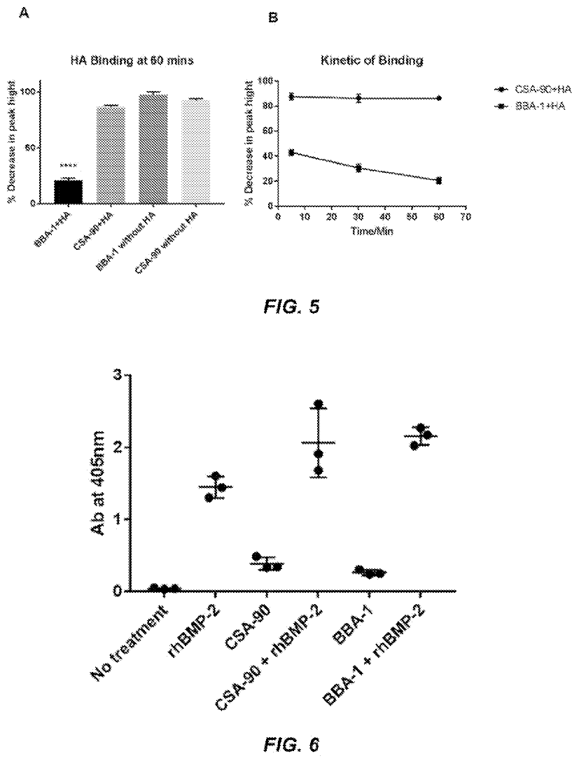

The relative peak height is attributable to BBA-1 decreased to ˜40% within 5 minutes of incubation with HA (FIG. 5-B). The most significant reduction was observed after 60 minutes of incubation, wherein the relative peak height of BBA-1 was decreased to ˜20% (FIG. 5-A). In contrast, CSA-90 showed no, or very low, reduction in peak height even after 60 minutes of incubation (FIG. 5A). After 60 minutes of incubation the corresponding peak height due to CSA-9 was only decreased to ˜85% (FIG. 5-A). These findings demonstrated that a significant amount of BBA-1 was bound to HA (P value ≤0.0001, unpaired t-test), and that the alendronate moiety conferred a high affinity to HA.

### Example 5: Measuring Effects on Osteogenic Activity

Parental compound CSA-90 has been reported to have pro-osteogenic activity and potentiate recombinant human bone morphogenetic protein-2 (rhBMP-2) in cultured cells (Schindeler et al., J Bone Joint Surg Am. 97(4):302-9 (2015)). This may lead to additional benefits beyond antimicrobial protection, particularly in the context of the repair of bony injuries, bone defects, or orthopedic implant osseointegration.

To test whether BBA-1 retained any of the pro-osteogenic action of CSA-90, MC3T3-E1 cells were differentiated in osteogenic media with either 5 μM CSA-90 or BBA-1, with or without 50 ng/ml rhBMP-2. A p-nitrophenyl phosphate assay was performed for alkaline phosphatase activity (Sigma-Aldrich) and normalized to day-3 cells grown in osteogenic media and α-MEM media. Assays were performed in triplicate with two independent repeats.

Consistent with prior published findings, CSA-90 and rhBMP-2 increased alkaline phosphatase expression (FIG. 6). BBA-1 showed similar osteogenic potential to CSA-90 both alone and in combination with rhBMP-2.

### Example 6: Measuring Impact on Mevalonate Pathway

Parental compound alendronate is a bisphosphonate that impacts on bone resorption by functionally affecting the mevalonate pathway. It was hypothesized by conjugation by the sidechain amine group, the bone binding affinity would be retained, but the anti-resorptive activity would be abrogated.

This was validated using an in vitro protein prenylation assay were performed on J774.2 monocyte macrophage lineage cells treated with range of doses of alendronate, CSA-90, or BBA-1. The assay used was as described by Ali N et al., Small GTPases 6(4):202-11 (2015). Briefly, this involved incubation of cells with the compounds for 24 hours at increasing doses of drugs and then an in vitro prenylation assay being performed on protein extracts. Immunoblotting (western blotting) was carried out to identify an exemplar unprenylated protein, with a band indicating drug activity affecting the mevalonate pathway.

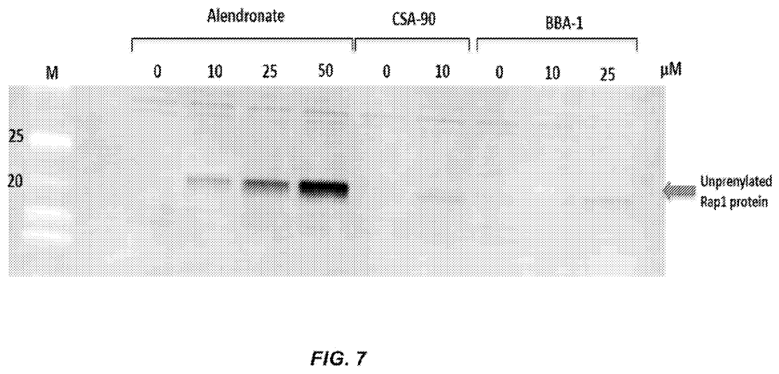

Treatment with alendronate at 25 μM concentration generated a potent effect on protein prenylation (FIG. 7). CSA-90 or BBA-1 treatment did not induce a noticeable or comparable protein prenylation effect at similar equimolar concentration of alendronate treatment. This indicates that alendronate conjugation to CSA-90 via its amine group considerably ameliorates its anti-osteoclastic activity.

### Example 7: Toxicity Study

Preliminary toxicity studies in mice have indicated that BBA-1 does not cause adverse events when given intravenously at a dose of 5 mg/kg.

In a proof-of-concept study mice were pre-dosed with 5 mg/kg BBA-1 one hour prior to surgery using a preclinical model of infection (N=10 per group). The model featured a drill hole made in the tibia of C57BL6 mice that was inoculated with live Staphylococcus aureus (strain ATCC 12600) at the time of surgery (1E5 CFU).

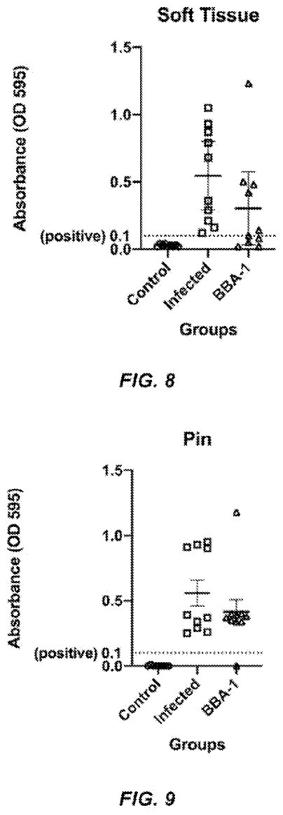

Control mice that did not receive infection did not test positive on swab assays of the soft tissue or pin. Numbers of infected samples and levels of infection as measured by optical density (OD595) when culturing for local infection (FIGS. 8 and 9) were reduced in the group treated with BBA-1 compared to the infected controls that did not receive BBA-1.

It will be appreciated by those skilled in the art that the compounds and methods described herein may be embodied in many other forms.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A compound having the following Formula (I) or a pharmaceutically acceptable salt form thereof:
B-L-C (I)
wherein:
B is a bone-binding moiety; L is a linker; and
C is a cationic steroid antimicrobial (CSA) moiety.

2. The compound of claim 1, wherein the CSA is selected from the group consisting of CSA-8, CSA-13, CSA-44, CSA-90, CSA-91, CSA-124, CSA-131, CSA-133, CSA-138, CSA-142, CSA-190, CSA-191, and CSA-192, such as where the CSA is CSA-13, CSA-90, or CSA-131, preferably CSA-90.

3. The compound of claim 1, wherein the bone-binding moiety is a bisphosphonate, such as where the bisphosphonate is selected from the group consisting of etidronate, clodronate, tiludronate, pamidronate, medronate, etidronate, neridronate, olpadronate, alendronate, ibandronate, aminomethylene diphosphonate, risedronate, and zoledronate, preferably the bisphosphonate is selected from the group consisting of alendronate, pamidronate and neridronate, more preferably the bisphosphonate is alendronate.

4. The compound of claim 1, wherein the linker is hydrophilic.

5. The compound of claim 1, wherein the linker has a molecular weight of less than about 2 kDa.

6. The compound of claim 1, wherein the linker comprises polyethylene glycol (PEG).

7. The compound of claim 1, wherein the linker has the following structure: 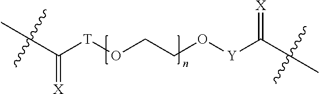
wherein:
X is independently selected from O and S;
T is absent or is an alkanediyl group having between 1 and 15 carbon atoms;
Y is absent or is an alkanediyl group having between 1 and 15 carbon atoms;
n is an integer between 1 and 30, and
the squiggly lines represent points of attachment to the CSA and bone-binding moieties.

8. The compound of claim 7, wherein X is O, T is an alkanediyl group having between 1 and 15 carbon atoms, Y is an alkanediyl group having between 1 and 15 carbon atoms, and n is an integer between 10 and 20.

9. The compound of claim 1, wherein the compound has the following structure: 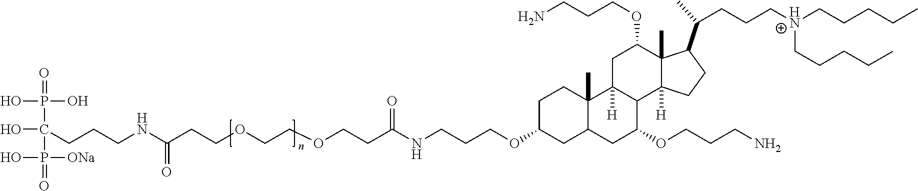
wherein n is between 1 and 50, such as wherein n is between 1 and 30 and wherein the compound has a molecular weight of between about 1.5 kDa and 2.5 kDa.

10. The compound of claim 1, wherein the compound has the following structure:

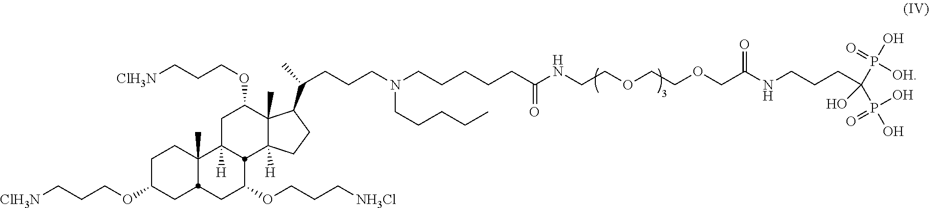

11. A pharmaceutical composition comprising the compound of claim 1 and a pharmaceutically acceptable carrier.

12. (canceled)

13. (canceled)

14. (canceled)

15. A method of treating an infection of a bone in a subject, the method comprising administering to the subject the compound of claim 1.

16. The method of claim 15, wherein the infection is a bacterial infection, wherein the bacterial infection is a Staphylococcus aureus infection, a Staphylococcus epidermidis infection, or a Pseudomonas aeruginosa infection.

17. The method of claim 15, wherein the bone comprises a fracture.

18. The method of claim 15, wherein the compound is administered systemically, orally, intravenously, or parenterally to the subject.

19. (canceled)

20. (canceled)

21. The method of claim 15, wherein the subject is a mammal.

22. A method of treating osteomyelitis in a subject, the method comprising administering to the subject the compound of claim 1.

23. The method of claim 22, wherein the osteomyelitis is associated with a Staphylococcus aureus infection, Staphylococcus epidermidis infection, or Pseudomonas aeruginosa infection.

24. The method of claim 22, wherein the compound is administered orally, intravenously, or parenterally to the subject.

25. (canceled)

26. The method of claim 22, wherein the subject is a human.

27. A method of promoting bone formation in a subject, the method comprising administering to the subject the compound of claim 1.

28. The method of claim 27, wherein the subject suffers from a bone disorder selected from the group consisting of a bone fracture, a spinal cord injury, spinal disc degeneration, Paget's disease, bone cancer, metastatic bone cancer, and osteoporosis.

29. The method of claim 27, wherein a bone of the subject is infected with one or more species of bacteria, including one or more of Staphylococcus aureus, Staphylococcus epidermidis, or Pseudomonas aeruginosa.

30. The method of claim 27, wherein the compound is administered systemically, orally, intravenously, or parenterally to the subject.

31-37. (canceled)
